(19) United States
(12) Patent Application Publication
(10) Pub. No.: US 2006/0167380 A1
(43) Pub. Date: Jul. 27, 2006

(54) Collection kit for obtaining specimens from urine

(76) Inventors: John Stanley Morton, Arvada, CO (US); Robert Peter Wills, Charleston, SC (US); Robert Dean Timm, Summerville, SC (US); James Blease Westmoreland, Charleston, SC (US)

Correspondence Address: HAMMER & HANF, PC, 3125 SPRINGBANK LANE, SUITE G, CHARLOTTE, NC, 28226, US

(21) Appl. No.: 11/043,316

(22) Filed: Jan. 26, 2005

(51) Int. Cl. A61B 5/00

(52) U.S. Cl. 600/573

(57) ABSTRACT

The instant application relates to a collection kit for obtaining specimens from urine. The collection kit, according to instant invention, includes a collection receptacle, a denaturizing agent, an acid, a delivery device, a first cartridge, a resin cartridge including a resin, and a sterile packaging device. The first cartridge is adapted to filter out organic compounds, while the resin possesses an affinity to bind a nuclide. The sterile packaging device contains the collection receptacle, the denaturizing agent, the acid, the delivery device, the first cartridge, and the resin cartridge.

### FIELD OF INVENTION

The instant application relates to a collection kit for obtaining specimens from urine.

### BACKGROUND OF THE INVENTION

It is generally necessary to collect biological fluids, e.g. urine, from a patient in order to determine whether such a person has been exposed to certain chemicals, e.g. radioactive materials such as depleted uranium. Generally, urine collected from a person of interest is stored in a disposable container, marked as biological fluids, and then transported to a laboratory for analysis. However, there is always the potential for contamination or loss of collected urine during the collection stage or the transportation stage; thus, careful measures must be employed to prevent any such contamination or loss of collected urine.

U.S. Pat. No. 4,953,561 discloses a urine antigen collection device. This device is in the form of a removable stackable sealed urine antigen sample container having an interior chamber with primary antibody covalently bonded to beads. The urine is pumped through the container where it engages and passes through a filter, which screens out cells and cell debris but allows passage of filtered urine fluid and antigen through an antibody bead bed. The beads in the bead bed have specific antibodies covalently bonded thereto to capture specific antigen carried by urine fluid. If there is an absence of the antigen in the specimen sample, the antibody will remain unoccupied and render a negative test result.

U.S. Pat. No. 4,960,130 discloses an apparatus for collecting biological fluids. This apparatus includes a tubular container having open ends, one of which is removeably secured to a collection storage unit. A shuttle assembly constructed of a cylindrical hollow piston defining a chamber, a top cover covering one end of the piston and a second cover with an aperture and a connector covering the second end of the piston is slideably mounted in the tubular container. An O-ring is mounted on the exterior surface of the piston to form a fluid tight seal between the O-ring and the interior surface of the tubular container with the connector being removeably secured to a resin/sample container so that movement of the piston in the tubular container carries the resin/sample container into the collection storage unit and forces fluid collected in the tubular container to flow through the resin/sample container.

U.S. Pat. No. 5,788,863 discloses a device for separating an analyte from an analyte-containing fluid. This device includes a body having a channel extending therethrough. The body includes a portion which is capable of receiving a fluid. The body also includes a fluid movement influencing portion which is coupled with a fluid moving means, such as a retractable plunger, to cause movement of the fluid through a membrane in both directions. Disposed between the fluid receiving portion and the fluid influencing portion is a membrane, which is capable of retaining an analyte from an analyte-containing fluid contacted therewith.

U.S. Pat. No. 5,976,824 discloses an apparatus for collecting a binding member of interest from a liquid specimen. This collection apparatus utilizes a collection receptacle, in which the specimen is deposited, with an affinity filter media being placed in communication with a discharge port in the collection receptacle, and a transfer device for drawing specimen from the collection receptacle and through the filter for capturing the binding member of interest. The collection receptacle, affinity-filter media and carrier therefore, and the transfer device may be supplied in kit form for use in a clinical environment.

However, there is still a need for a collection kit, which facilitates the collection and transportation of urine specimens to a laboratory for analysis while diminishing the possibility of leakage or contamination of the collected urine sample.

### SUMMARY OF THE INVENTION

The instant application relates to a collection kit for obtaining specimens from urine. The collection kit, according to instant invention, includes a collection receptacle, a denaturizing agent, an acid, a delivery device, a first cartridge, a resin cartridge including a resin, and a sterile packaging device. The first cartridge is adapted to filter out organic compounds, while the resin possesses an affinity to bind a nuclide of interest. The sterile packaging device contains the collection receptacle, the denaturizing agent, the acid, the delivery device, the first cartridge, and the resin cartridge.

### BRIEF DESCRIPTION OF THE DRAWINGS

For the purpose of illustrating the invention, there is shown in the drawings a form that is presently preferred; it being understood, however, that this invention is not limited to the precise arrangements and instrumentalities shown.

FIG. 1 is a first embodiment of the instant invention;

FIG. 2 is a second embodiment of the instant invention;

FIG. 3 is a schematic illustration of how the first embodiment of the instant invention operates; and

FIG. 4 is a schematic illustration of how the second embodiment of the instant invention operates.

### DETAILED DESCRIPTION OF THE INVENTION

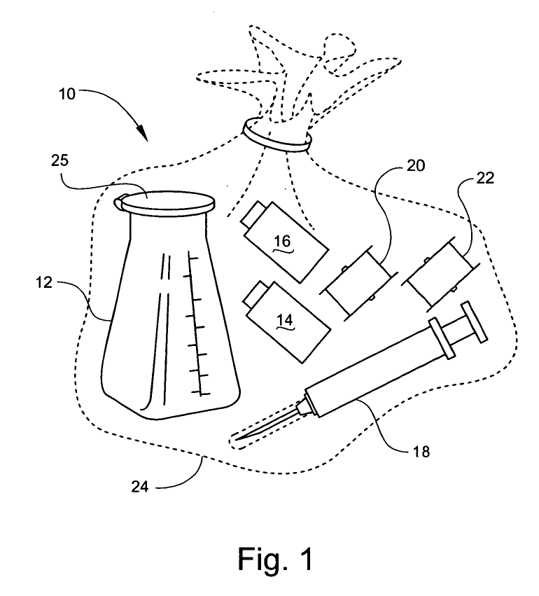

Referring to the drawings wherein like numerals indicate like elements, there is shown, in FIG. 1, a collection kit 10 for obtaining specimen from urine according to the first embodiment of instant invention. Collection kit 10 includes a collection receptacle 12, denaturizing agent 16, acid 14, delivery vehicle 18, first cartridge 20, resin cartridge 22, and a sterile packaging device 24.

Collection receptacle 12 may be made from any material, and may have any shape or size. For example, collection receptacle 12 may be made of a polymer, e.g. polyethylene, or a metal. Collection 12 may also include a cap 25 to prevent the spillage of collected urine. Collection receptacle 12 may, for example, have a cylindrical shape, and a size in the range of about 10 ml to about 2000 ml. Collection receptacle 12 is further adapted to facilitate the collection of urine from a person of interest.

Denaturizing agent 16 may be any denaturing agent. Denaturizing agent 16 is adapted to facilitate the denaturization of proteins, i.e. breaking down the protein chains; hence, allowing better access to inorganic nuclides of interest. For example, denaturizing agent 16 may be selected from the group consisting of sodium dodecyl sulfate, urea, and chlorhexidine. Denaturizing agent 16 may further be stored, for example, in a vial, a container, or the like. Collection kit 10 may have any amount of denaturizing agent 16; for example, collection kit 10 may have between about 0.1 ml to about 1.0 ml of the denaturizing agent 16. Such denaturizing agents 16 are commercially available under the name of sodium lauryl sulfate from J.T. Baker or Sigma Aldrich of New Jersey.

Acid 14 may be any acid. For example, acid 14 may be selected from the group consisting of hydrochloric acid and nitric acid. Acid 14 may have any concentration; for example, acid 14 may have a concentration in the range of about 6M to about 12M. Acid 14 may further be stored, for example, in a vial, a container, or the like. Collection kit 10 may have any amount of acid 14; for example, collection kit 10 may have between about 1 ml to about 20 ml of the acid 14. Such acids 14 are commercially available under the name of nitric acid from J.T. Baker of New Jersey.

Delivery vehicle 18 may have any shape or any volume. Delivery vehicle 18 may, for example, be a syringe. Delivery vehicle 18 is further adapted to facilitate the separation of the specimen of interest from the collected urine. Furthermore, delivery vehicle 18 may have a volume in the range of about 1 ml to about 1000 ml. Such delivery vehicles 18 are commercially available under the name of Monject from B-D Plastic of New Jersey.

First cartridge 20 may be a filtration device. First cartridge 20 may be adapted to filter out organic compounds such as proteins commonly found in urine. Such first cartridges 20 are commercially available under the name of Pre-Filter Resin from Eichrom of Illinois.

Resin cartridge 22 may be any cartridge including a resin. For example, resin cartridge 22 may include a resin having an affinity for a specific nuclide selected from the group consisting of uranium, iodine, strontium, and mixed-fusion and activation products. Resin, as used herein, refers to any solid or semisolid material having an affinity for a specific nuclide. Affinity for a specific nuclide, as used herein, refers to the tendency of the resin to combine with a specific nuclide. Such resin cartridges 22 are commercially available under the name of UTEVA-2 from Eichrom of Illinois.

Sterile packaging device 24 may be any packaging device. For example, sterile packaging device 24 may be a packaging device selected from the group consisting of a sealed container, a plastic wrap, a case, a box, a bag, and the like.

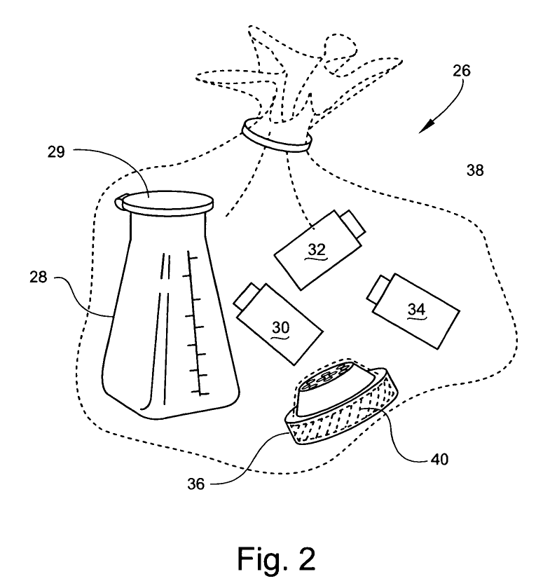

Referring to FIG. 2, there is shown an alternative embodiment of the kit for collection of specimen from urine according to instant invention. Collection kit 26 includes a collection receptacle 28, a separation device 36, a denaturizing agent 30, an acid 32, a resin 34, and a sterile packaging device 38.

Collection receptacle 28 may be made from any material, and may have any shape or size. For example, collection receptacle 28 may be made of a polymer, e.g. polyethylene, or a metal. Collection 28 may also include a cap 29 to prevent the spillage of collected urine. Collection receptacle 28 may, for example, have a cylindrical shape, and a size in the range of about 10 ml to about 2000 ml. Collection receptacle 28 is further adapted to facilitate the collection of urine from a person of interest.

Separation device 36 may be any device suitable for separating the nuclide-resin-complex of interest from urine. For example, the separation device 36 may be a modified cap including a filter 40, e.g. a filter on the inside of a short column with threads allowing the device to be closed at both ends for sterile shipping. The filter 40 may be made of any material. For example, filter 40 may be polymers, glass fibers, sinistered glass, and the like. Separation device 36 is further adapted to be affixed to the opening of collection receptacle 28.

Denaturizing agent 30 may be any denaturing agent. Denaturizing agent 16 is adapted to facilitate the denaturization of proteins, i.e. breaking down the protein chains; hence, allowing better access to inorganic nuclides of interest. For example, denaturizing agent 30 may be selected from the group consisting of sodium dodecyl sulfate, urea, and chlorhexidine. Denaturizing agent 30 may further be stored, for example, in a vial, a container, or the like. Collection kit 26 may have any amount of denaturizing agent 30; for example, collection kit 26 may have between about 0.1 ml to about 3.0 ml of denaturizing agent 30. Such denaturizing agents 30 are commercially available under the name of sodium lauryl sulfate from J.T. Baker or Sigma Aldrich of New Jersey.

Acid 32 may be any acid. For example, acid 32 may be selected from the group consisting of hydrochloric acid and nitric acid. Acid 32 may have any concentration; for example, acid 32 may have a concentration in the range of about 6M to about 12M. Acid 32 may further be stored, for example, in a vial, a container, or the like. Collection kit 26 may have any amount of acid 32; for example, collection kit 26 may have between about 1 ml to about 20 ml of acid 32. Such acids 32 are commercially available under the name of nitric acid from J.T. Baker or Sigma Aldrich of New Jersey.

Resin 34 may be any material. Resin 34 may be a solid or semisolid material having an affinity for a specific nuclide. For example, resin 34 may include a resin having an affinity for a specific nuclide selected from the group consisting of uranium, iodine, strontium, and mixed-fission and activation products. Affinity for a specific nuclide, as used herein, refers to the tendency of the resin to combine with a specific nuclide. Resin 34 may further be stored, for example, in a vial, a container, or the like. Collection kit 26 may have any amount of resin 34; for example, collection kit 26 may have between about 1 ml to about 5 ml of resin 34. Such resins 34 are commercially available under the name of UTEVA-2 from Eichrom of Illinois.

Sterile packaging device 38 may be any packaging device. For example, sterile packaging device 38 may be a packaging device selected from the group consisting of a sealed container, a plastic wrap, a case, a box, a bag, and the like.

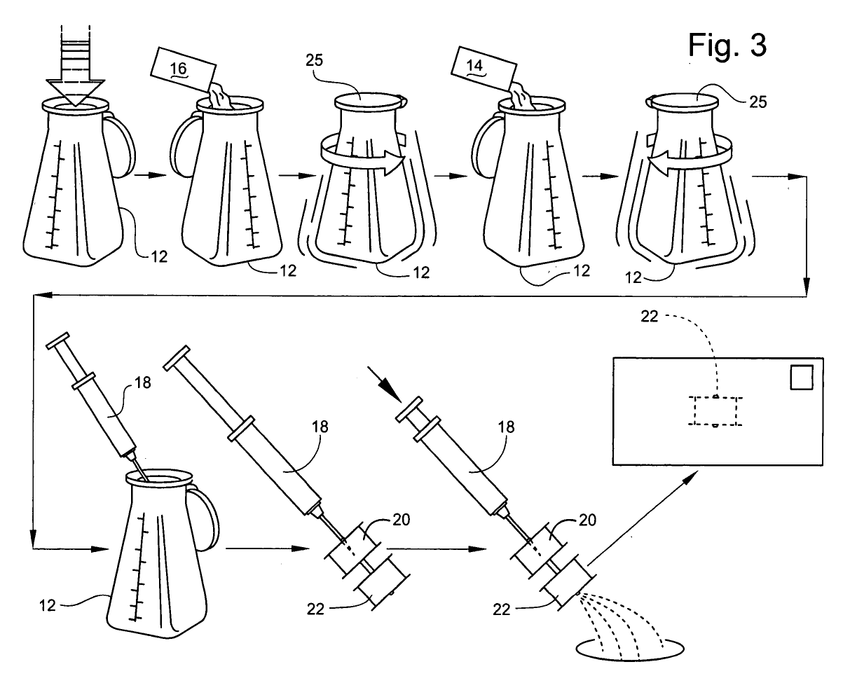

In operation, referring to FIG. 3, urine sample is collected in the collection receptacle 12; the denaturizing agent 16 is added to the urine sample; the collection receptacle 12 is sealed via a cap 25; and then, the content of the collection receptacle 12, which includes the urine sample and the denaturizing agent 16, is mixed by swirling for a period of about five (5) minutes to about ten (10) minutes. Subsequently, the cap 25 is removed; acid 14 is added to the admixture of the urine sample and the denaturizing agent 16; collection receptacle 12 is sealed via the cap 25; and then, the content of the collection receptacle 12, which includes acid 14 and the admixture of the urine sample and the denaturizing agent 16, is mixed by swirling for a period of about five (5) minutes to about ten (10) minutes. Finally, the cap 25 is removed; an appropriate amount of the admixture of the urine sample, the denaturizing agent 16, and the acid 14 is transferred into the delivery vehicle 18 (e.g. syringe); first cartridge 20 and resin cartridge 22 are adjoined to the delivery vehicle 18, and then the admixture of the urine sample, the denaturizing agent 16, and the acid 14 is forced out of the delivery vehicle 18 via first cartridge 20 and resin cartridge 22. The first cartridge 20 removes any undesired organic compounds while resin cartridge 22 collects the nuclide of interest. Resin cartridge 22 may be sealed, and then, transported to a laboratory for further analysis.

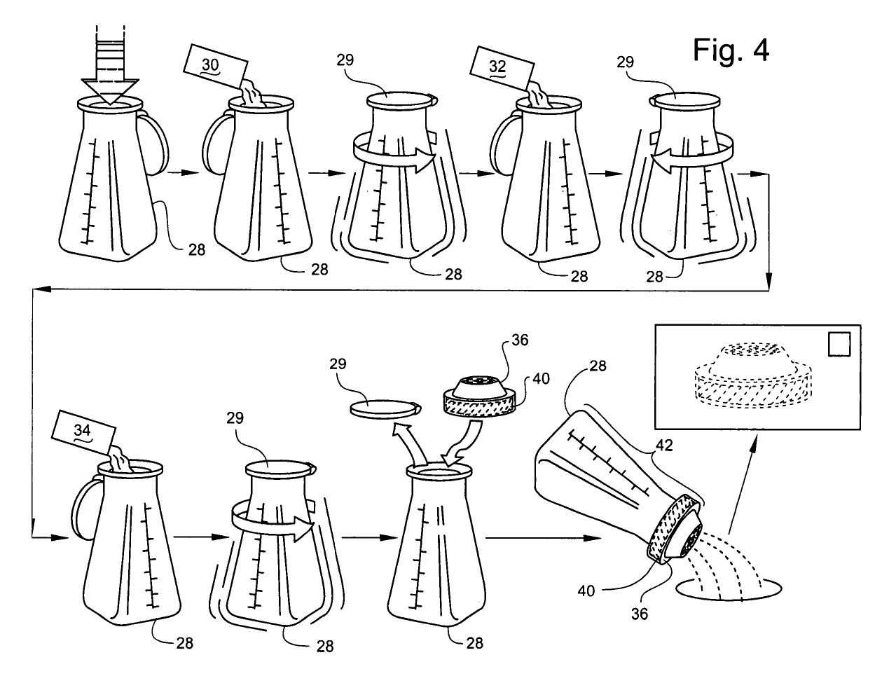

In the alternative operation, referring to FIG. 4, urine sample is collected in the collection receptacle 28; the denaturizing agent 30 is added to the urine sample; the collection receptacle 28 is sealed via a cap 29; and then, the content of the collection receptacle 28, which includes the urine sample and the denaturizing agent 30, is mixed by swirling for a period of about five (5) minutes to about ten (10) minutes. Subsequently, the cap 29 is removed; acid 32 is added to the admixture of the urine sample and the denaturizing agent 30; collection receptacle 28 is sealed via the cap 29; and then, the content of the collection receptacle 28, which includes acid 32 and the admixture of the urine sample and the denaturizing agent 30, is mixed by swirling for a period of about five (5) minutes to about ten (10) minutes. Finally, the cap 29 is removed; resin 34 is added to the admixture of the urine sample, denaturizing agent 30, and acid 32; collection receptacle 28 is sealed via the cap 29; the content of the collection receptacle 28, which includes resin 34, and the admixture of the urine sample, the denaturizing agent 30, and the acid 32, is mixed by swirling for a period of about five (5) minutes to about ten (10) minutes allowing the resin 34 to form a complex with the nuclide of interest; the cap 29 is replaced with the separation device 36; and then, the collection receptacle-separation device-assembly 42 is inverted thereby permitting the urine out of the collection receptacle 28 while collecting rein-nuclide-complex of interest. Separation device 36 may be sealed, and then, transported to a lab for further analysis.

The present invention may be embodied in other forms without departing from the spirit and the essential attributes thereof, and, accordingly, reference should be made to the appended claims, rather than to the foregoing specification, as indicated the scope of the invention.

We claim:

1. A collection kit for obtaining specimens from urine comprising:
a collection receptacle;
a denaturizing agent;
an acid;
a delivery device;
a first cartridge, said first cartridge adapted to filter out organic compounds;
a resin cartridge including a resin, said resin having an affinity to bind a nuclide; and
a sterile packaging device, said sterile packaging device containing said collection receptacle, said acid, said denaturizing agent, said delivery device, said first cartridge, and said resin cartridge.

2. The collection kit for obtaining specimens from urine according to claim 1, wherein said denaturizing agent being selected from the group consisting of sodium dodecyl sulfate, urea, and chlorhexidine.

3. The collection kit for obtaining specimens from urine according to claim 1, wherein said collection kit having 0.1 ml to 1.0 ml of said denaturizing agent.

4. The collection kit for obtaining specimens from urine according to claim 1, wherein said acid being selected from the group consisting of hydrochloric acid and nitric acid.

5. The collection kit for obtaining specimens from urine according to claim 1, wherein said collection kit having 0.1 ml to 20.0 ml of said acid.

6. The collection kit for obtaining specimens from urine according to claim 1, wherein said delivery device being a syringe.

7. The collection kit for obtaining specimens from urine according to claim 6, wherein said syringe having a volume in the range of 1 ml to 100 ml.

8. The collection kit for obtaining specimens from urine according to claim 1, wherein said first cartridge being capable of filtering out organic compounds.

9. The collection kit for obtaining specimens from urine according to claim 1, wherein said resin having an affinity to bind a nuclide selected from the group consisting of uranium, iodine, strontium, and mixed-fission and activation products.

10. The collection kit for obtaining specimens from urine according to claim 1, wherein said sterile packaging device being selected from the group consisting of a sealed container, a plastic wrap, a case, a box, a bag, and the like.

11. A collection kit for obtaining specimens from urine comprising:
a collection receptacle;
separation device;
a denaturizing agent;
an acid;
a resin having an affinity to bind a nuclide; and
a sterile packaging device, said sterile packaging device containing said collection receptacle, said separation device, said acid, said denaturizing agent, and said resin.

12. The collection kit for obtaining specimens from urine according to claim 11, wherein said separation device being a modified cap including a filter therein.

13. The collection kit for obtaining specimens from urine according to claim 11, wherein said denaturizing agent being selected from the group consisting of sodium dodecyl sulfate, urea, and chlorhexidine.

14. The collection kit for obtaining specimens from urine according to claim 11, wherein said collection kit having 0.1 ml to 1.0 ml of said denaturizing agent.

15. The collection kit for obtaining specimens from urine according to claim 11, wherein said acid being selected from the group consisting of hydrochloric acid and nitric acid.

16. The collection kit for obtaining specimens from urine according to claim 11, wherein said collection kit having 0.1 ml to 20.0 ml of said acid.

17. The collection kit for obtaining specimens from urine according to claim 11, wherein said resin having an affinity to bind a nuclide selected from the group consisting of uranium, iodine, strontium, and mixed-fission and activation products.

18. The collection kit for obtaining specimens from urine according to claim 11, wherein said sterile packaging device being selected from the group consisting of a sealed container, a plastic wrap, a case, a box, a bag, and the like.
